(19) United States
(12) Patent Application Publication
(10) Pub. No.: US 2015/0079160 A1
(43) Pub. Date: Mar. 19, 2015

(54) NON-IMPLANTABLE MEDICAL DEVICE COATED WITH NANO-CARRIERS FOR DELIVERING ONE OR MORE DRUGS TO A BODY SITE

(71) Applicant: Concept Medical Research Private Limited, Nanpura (IN)

(72) Inventors: MANISH DOSHI, Surat (IN); Prakash Sojitra, Surat (IN); Pedro A Lemos Neto, Sao Paulo (BR)

(73) Assignee: CONCEPT MEDICAL RESEARCH PRIVATE LIMITED, Nanpura (IN)

(21) Appl. No.: 14/458,138

(22) Filed: Aug. 12, 2014

Related U.S. Application Data
(63) Continuation-in-part of application No. 12/920,812, filed on Sep. 2, 2010, now Pat. No. 8,801,662.

(30) Foreign Application Priority Data
Jun. 2, 2009 (IN) 1337/MUM/2009

(51) Int. Cl. A61L 29/08; A61K 31/436; A61K 31/337

(52) U.S. Cl. CPC A61L 29/08; A61K 31/337; A61K 31/436
USPC 424/450; 424/400; 514/449; 514/291; 424/451

(57) ABSTRACT

A drug-delivering medical device for delivering a drug to a target site in a human body is disclosed. The drug-delivering medical device may have a hydrophilic surface, with one or more portions of the hydrophilic surface coated with one or more nano-carriers bearing one or more drugs. Each nano-carrier may include a drug surrounded by an encapsulating medium. As the drug is surrounded by the encapsulating medium, the surface of each nano-carrier can be devoid of the respective drug. A non-implantable medical device coated with nano-carriers can deliver one or more drugs to a blood vessel, organ cavity, sac, capsule, lining, layer, coating, membrane, connective tissue, fluid surrounding an organ, and so forth.

### RELATED APPLICATIONS

This continuation-in-part application claims the benefit of priority to U.S. patent application Ser. No. 12/920,812, filed Sep. 2, 2010, now U.S. Pat. No. 8,801,662, which is a National Phase Application of International PCT Application No. PCT/IN2010/000349, filed May 21, 2010, which claims priority to India Patent Application No. 1337/MUM/2009, filed Jun. 2, 2009.

### FIELD OF THE INVENTION

The invention generally relates to a medical device for administrating a drug to a target site in the body, such as a blood vessel, organ cavity, sac, capsule, lining, layer, coating, membrane, connective tissue, fluid surrounding an organ, and so forth. More specifically, the invention relates to a drug-delivering instrument coated with nano-carriers of one or more drugs for efficient delivery of the one or more drugs to the target site in the body.

### BACKGROUND OF THE INVENTION

Drug-delivering insertable medical devices are used for localized delivery of a drug to a target site in a blood vessel. Drug Eluting Balloon (DEB) is one such drug-delivering insertable medical device. Although, widely used, the DEBs are associated with use of polymers for loading the drug on the surface of the DEBs. The polymers used may cause inflammation at the target site. To avoid the inflammation caused by the polymers, the drug may be loaded on the surface of the DEB without using the polymers. However, such polymer-free approaches known in the art are based on surface modification of the DEB.

Additionally, the DEB comes in contact with the target site in the blood vessel for a very short period, generally, ranging from 30 seconds to 90 seconds. The required amount of the drug loaded on the DEB may not be released from the surface of the DEB within such a short period. Therefore, a higher amount of the drug has to be loaded on the DEB as compared to the amount of the drug actually required to be delivered. As the higher amount of the drug is loaded on the DEB, a substantial amount of the drug may remain on the surface of the DEB after the DEB is withdrawn from the target site. The remaining amount of the drug present on the DEB may get washed away in the blood stream while the DEB is being withdrawn through the blood vessel thereby producing unwanted side effects.

Further, micro-sized drug particles are coated on the current DEBs. The micro-sized particles of the drug may not penetrate tissues at the target site efficiently. Thus, the current DEBs may not exhibit an efficient in-tissue diffusion of the drug.

Therefore, there is a need in the art for a DEB that can efficiently deliver the drug to the target site in the blood vessel within a short period for which the DEB comes in contact with the target site. In addition, there is a need in the art for a DEB that can efficiently deliver drug to the maximum area of a lesion and provide for enhance bioavailability with an optimum amount of the drug loaded on the drug-delivering balloon.

### BRIEF DESCRIPTION OF FIGURES

In the accompanying figures, like reference numerals refer to identical or functionally similar elements throughout the separate views and which together with the detailed description below are incorporated in and form part of the specification, serve to further illustrate various embodiments and to explain various principles and advantages all in accordance with the invention.

FIG. 1 illustrates the size distribution of nano-particles of soya phospholipid as detected by Malvern ZS90 in accordance with Example 1.

FIG. 2 illustrates the size distribution of nano-carriers as detected by Malvern ZS90 in accordance with Example 1.

FIG. 3 is a table illustrating numbers assigned to 17 animals, stent type and location of stent in each animal and the type of study (PK/LM) conducted on the animals in accordance with Example 2.

FIG. 4 illustrates a table of values of mean and standard derivations of the Average Luminal Areas, Average Stent Areas, Average Neointimal Areas and Average Percent Neointimal Obstruction findings for each group i.e. Control, Sirolimus and Paclitaxel in accordance with Example 3.

FIG. 5 illustrates a table of values of mean and standard derivations of the Median Luminal Areas, Median Stent Areas, Median Neointimal Areas and Median Percent Neointimal Obstruction findings for each group in accordance with Example 3.

FIG. 6 illustrates a table of values of mean and standard derivations of the Minimal Luminal Areas, Minimal Stent Areas, Minimal Neointimal Areas and Minimal Percent Neointimal Obstruction findings for each group in accordance with Example 3.

FIG. 7 illustrates a table of values of mean and standard derivations of the Maximal Luminal Areas, Maximal Stent Areas, Maximal Neointimal Areas and Maximal Percent Neointimal Obstruction findings for each group in accordance with Example 3.

FIG. 8 is a diagram of a drug-delivering medical device shown in a body lumen, that includes a balloon catheter, an inflatable balloon positioned on the balloon catheter, the inflatable balloon having a hydrophilic surface, at least one portion of the hydrophilic surface being coated with a plurality of nano-carriers (nano-carriers not shown to scale), with at least one of the nano-carriers surrounded by an encapsulating medium.

FIG. 9 is a diagram of example PK results from rabbit iliac artery.

FIG. 10 is a diagram of example drug remaining of the balloon post in-vivo treatment.

FIG. 11 is a diagram of example tissue concentration at an eight day time point.

FIG. 12 is a diagram of example confocal analysis for DTF-labeled nano particle sirolimus. Image (A) shows an example en face microscopy; image (B) shows an example cross-section.

FIG. 13. is a diagram showing example en face images of the surface of a balloon used for treatment.

FIG. 14 is a diagram showing an example luminal surface of a treated iliac artery with DTF-nSRL at one hr; image (A) shows an en face image; image (B) shows an example cross-section (L=lumen, M=medial layer, IEL=internal elastic lamina).

FIG. 15 is a diagram showing an example luminal surface of a treated iliac artery with DTF-nSRL at 24 hours; image (A) shows an example en face image; image (B) shows an example cross-section. (L=lumen, M=medial layer, IEL=internal elastic lamina)

FIG. 16 is a diagram of an example luminal surface of a treated iliac artery with DTF-nSRL at three days; image (A) shows an example en face image; image (B) shows an example cross-section. (L=lumen, M=medial layer, IEL=internal elastic lamina)

FIG. 17 is a diagram of an example luminal surface of a treated iliac artery with DTF-nSRL at 7 days; image (A) shows an example en face image; image (B) shows an example cross-section. (L=lumen, M=medial layer, IEL=internal elastic lamina)

FIG. 18 is a table of DTF-nSRL penetration depth in the arterial layer.

FIG. 19 shows SEM images of an example sirolimus nanoparticles coated balloon; image (A) shows 225X; image (B) shows 1KX; image (C) shows 10KX; image (D) shows 30KX.

FIG. 20 is a diagram showing, in image (A) example particle size analysis by Malvern ZS 90; image (B) shows an example SEM image of sirolimus nanoparticles.

### DETAILED DESCRIPTION OF THE INVENTION

Before describing in detail embodiments that are in accordance with the invention, it should be observed that the embodiments reside primarily in combinations of components of a drug-delivering insertable medical device for localized delivery of a drug to a target site in a blood vessel. Accordingly, the components have been described to include only those specific details that are pertinent to understanding the embodiments of the invention so as not to obscure the disclosure with details that will be readily apparent to those of ordinary skill in the art having the benefit of the description herein.

In this document, the terms “comprises”, “comprising” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element preceded by “comprises . . . a” does not, without more constraints, preclude the existence of additional identical elements in the process, method, article, or apparatus that comprises the element.

Further, before describing in detail embodiments that are in accordance with the invention, it should be observed that all the scientific and technical terms used herein for describing the invention have same meanings as would be understood by a person skilled in the art.

Generally speaking, pursuant to various embodiments, the invention discloses a drug-delivering medical device for delivering a drug to a target site in a body lumen. The body lumen may be one of a blood vessel, a urethra, an esophagus, a ureter and a bile duct. The drug-delivering medical device is used to treat a medical condition associated with the body lumen. The medical condition may be, for example, atherosclerosis, a blocked artery, restenosis, plaque accumulation in a blood vessel and thrombus formation in the blood vessel.

The drug-delivering medical device includes a balloon catheter and an inflatable balloon positioned on the balloon catheter. The inflatable balloon has a hydrophilic surface. One or more portions of the hydrophilic surface are coated with two or more nano-carriers. A nano-carrier of the two or more nano-carriers includes a drug surrounded by an encapsulating medium. The encapsulating medium may be one or more of a biological agent, a blood excipient and a phospholipid. As the drug is surrounded by the encapsulating medium, the surface of the nano-carrier is devoid of the drug. When the inflatable balloon is inflated upon coming in proximity to the target site in the body lumen, about 30% to 80% of the two or more nano-carriers are released from the hydrophilic surface within 15 to 90 seconds.

The drug-delivering medical device may be a balloon catheter assembly generally used for Percutaneous Transluminal Angioplasty (PTA). In an embodiment, the drug-delivering medical device is a balloon catheter assembly generally used for Percutaneous Transluminal Coronary Angioplasty (PTCA). The balloon catheter assembly essentially includes a balloon catheter and an inflatable balloon mounted on the balloon catheter. Additionally, the balloon catheter assembly may include one or more catheter tubes, one or more guide wires, a fluid supply for inflating the inflatable balloon, and any other component that may be necessary for functioning of the balloon catheter assembly for delivering the two or more nano-carriers to the target site. As the invention primarily resides in the inflatable balloon coated with the two or more nano-carriers, the functioning and components of the balloon catheter assembly other than the inflatable balloon are not disclosed in detail.

The inflatable balloon may be any balloon that is generally used in one of the PTA and the PTCA and that may also be used to deliver the two or more nano-carriers to the target site in the body lumen. For example, the inflatable balloon may be an angioplasty balloon.

The inflatable balloon has a hydrophilic surface. The hydrophilic surface may be a layer of one or more hydrophilic substances coated on the surface of the inflatable balloon, generally, to reduce the friction of the inflatable balloon with the walls of the body lumen when the inflatable balloon is moved in the body lumen. In accordance with various embodiments, the one or more hydrophilic substances may be selected from the hydrophilic substances known in the art without departing from the scope of the invention. For example, the one or more hydrophilic substances may be selected from, one or more of, but are not limited to, polyalkylene glycols, alkoxy polyalkylene glycols, copolymers of methylvinyl ether and maleic acid poly(vinylpyrrolidone), poly(N-alkylacrylamide), poly(acrylic acid), poly(vinyl alcohol), poly(ethyleneimine), methyl cellulose, carboxymethyl cellulose, polyvinyl sulfonic acid, heparin, dextran, modified dextran and chondroitin sulphate and at least one antiblock agent.

One or more portions of the hydrophilic surface are coated with the two or more nano-carriers. The two or more nano-carriers coated on the one or more portions of the hydrophilic surface are released rapidly as compared to the release of nano-carriers from the surface of a balloon without hydrophilic coating. Thus, a burst release of the two or more nano-carriers from the hydrophilic surface may be achieved within a short period for which the inflatable balloon comes in contact with the target site in the body lumen. In an exemplary embodiment, about 70% to 80% of the two or more nano-carriers are released from the hydrophilic surface within about 60 seconds when the inflatable balloon is inflated upon coming in proximity to the target site.

According to various embodiments, the hydrophilic surface may further have one or more exposed hydrophilic surfaces. The one or more exposed hydrophilic surfaces may be created at one or more desired portions of the inflatable balloon by not coating the two or more nano-carriers on the one or more desired portions. In other words, the two or more nano-carriers are coated on the entire hydrophilic surface except the one or more exposed hydrophilic surfaces. In an embodiment, the one or more exposed hydrophilic surfaces are created at one or more of one or more portions of a distal end of the hydrophilic surface and one or more portions of a proximal end of the hydrophilic surface. The one or more exposed hydrophilic surfaces facilitate dissolution of the one or more hydrophilic substances present in the one or more exposed hydrophilic surfaces upon coming in contact with the blood at the target site. In response to the dissolution of the one or more hydrophilic substances present in the one or more exposed hydrophilic surfaces, the one or more hydrophilic substances present in the hydrophilic surface may also get dissolved in the blood resulting into release of the two or more nano-carriers from the hydrophilic surface. Thus, the one or more exposed hydrophilic surfaces facilitate the release of the two or more nano-carriers from the inflatable balloon.

The inflatable balloon further has two or more nano-carriers coated on the one or more portions of the hydrophilic surface. A nano-carrier of the two or more nano-carriers includes a drug surrounded by an encapsulating medium. The drug may include nano-crystals of the drug. The nano-crystals of the drug may have an average diameter ranging from 1 nm to 1500 nm. Further, the nano-crystals of the drug may have two or more different average diameters. Alternatively, the drug may include one or more of, nano-sized particles, nano-spheres, liposomes, nano-capsules, dendrimers, and any other similar form of the drug that has nano-dimensions.

The drug may be, one or more of, but are not limited to, an anti-proliferative agent, an anti-inflammatory agent, an anti-neoplastic agent, an anti-coagulant agent, an anti-fibrin agent, an antithrombotic agent, an anti-mitotic agent, an antibiotic agent, an anti-allergic agent and an antioxidant, an anti-proliferative agent, estrogens, a protease inhibitor, antibodies, an immunosuppressive agent, a cytostatic agent, a cytotoxic agent, a calcium channel blocker, a phosphodiesterase inhibitor, a prostaglandin inhibitor, a dietary supplement, vitamins, anti-platelet aggregating agent and genetically engineered epithelial cells.

The drug may be, for example, but are not limited to, one or more of sirolimus, tacrolimus, paclitaxel, clobetasol, dexamethasone, genistein, heparin, 17 beta-estadiol, rapamycin, everolimus, ethylrapamycin, zotarolimus, ABT-578, Biolimus A9, docetaxel, methotrexate, azathioprine, vincristine, vinblastine, fluorouracil, doxorubicin hydrochloride, mitomycin, sodium heparin, a low molecular weight heparin, a heparinoid, hirudin, argatroban, forskolin, vapiprost, prostacyclin, a prostacyclin analogue, dextran, D-phe-pro-arg-chloromethylketone, dipyridamole, glycoprotein IIb/IIIa, recombinant hirudin, bivalirudin, nifedipine, colchicines, lovastatin, nitroprusside, suramin, a serotonin blocker, a steroid, a thioprotease inhibitor, triazolopyrimidine, a nitric oxide or nitric oxide donor, a super oxide dismutase, a super oxide dismutase mimetic, estradiol, aspirin, angiopeptin, captopril, cilazapril, lisinopril, permirolast potassium, alpha-interferon, bioactive RGD and any salts or analogues thereof.

In accordance with various embodiments, the drug is completely surrounded by the encapsulating medium and thus the surface of the nano-carrier is devoid of any free drug. Therefore, a direct contact of the drug with the surface of the inflatable balloon is avoided. Further, the drug comes in contact with the tissues of the target site only when the nano-carrier penetrates into tissues of the target site and the encapsulating medium is dissolved. Thus, direct exposure of the drug to the tissues of the target site and the surface of the inflatable is prevented due to the presence of the encapsulating medium.

The encapsulating medium may be one or more of, a biological agent, a blood excipient and a phospholipid. Alternatively, the encapsulating medium may be one or more of, but not are limited to, one or more biological agents, one or more blood excipients, one or more phospholipids and one or more excipients.

In an exemplary embodiment, the encapsulating medium may be a biological agent. The biological agent may include nano-particles of the biological agent. The nano-particles of the biological agent may have an average diameter ranging from 1 nm to 1500 nm. Further, the nano-particles of the biological agent may have two or more different average diameters. Alternatively, the biological agent may include one or more of, nano-sized particles, nano-spheres, liposomes, nano-capsules, dendrimers, and any other similar form of the biological agent that has nano-dimensions.

The biological agent may be one or more of, but are not limited to, drug carriers, excipients, blood components, excipients derived from blood, naturally occurring phospholipids, solid lipid nano-particles, phospholipids obtained from a living animal, synthetically derived phospholipids, lipoids, vitamins and sugar molecules. The biological agent may include, for example, steroids, vitamins, estradiol, esterified fatty acids, non esterified fatty acids, glucose, inositol, L-lactate, lipoproteins, carbohydrates, tricalcium phosphate, precipitated calcium phosphate, calcium phoshate tribasic, substances derived from at least one of human, egg and soybean, phospholipon 80H, phospholipon 90H, Lipoids S75, Lipoids E80, Intralipid 20, Lipoid EPC, Lipoids E75, lipids obtained from egg, lipids obtained from soya, phosphatidylcholine, phosphatidylglycerol, phosphatidylinositol, phosphatidylserine, phosphatidic acid, cardiolipin, and phosphatidylethanolamine.

In another exemplary embodiment, the encapsulating medium may be a phospholipid. The phospholipid may include nano-particles of the phospholipid. The nano-particles of the phospholipid may have an average diameter ranging from 1 nm to 1500 nm. Further, the nano-particles of the phospholipid may have two or more different average diameters. Alternatively, the phospholipid may include one or more of, nano-sized particles, nano-spheres, liposomes, nano-capsules, dendrimers, and any other similar form of the phospholipid that has nano-dimensions. The phospholipid may be one or more of, but are not limited to, lipids obtained from egg, lipids obtained from soya, phosphatidylcholine, phosphatidylglycerol, phosphatidylinositol, phosphatidylserine, phosphatidic acid, cardiolipin, and phosphatidylethanolamine.

In yet another exemplary embodiment, the encapsulating medium may be a blood excipient. The blood excipient may include nano-particles of the blood excipient. The nano-particles of the blood excipient may have an average diameter ranging from 1 nm to 1500 nm. Further, the nano-particles of the blood excipient may have two or more different average diameters. Alternatively, the blood excipient may be, one or more of, nano-sized particles, nano-spheres, liposomes, nano-capsules, dendrimers, and any other similar form of the blood excipient that has nano-dimensions. The blood excipient may be one or more excipients found in the blood that may be manufactured synthetically. The blood excipient may be one or more of, but are not limited to, steroids, vitamins, estradiol, esterified fatty acids, non-esterified fatty acids, glucose, inositol, L-lactate, lipids, lipoproteins, phospholipid, carbohydrates, tricalcium phosphate, precipitated calcium phosphate, and calcium phoshate tribasic.

The blood excipient and the biological agent are soluble at a pH below 7.4. Therefore, when the two or more nano-carriers come in contact with the tissues of the target site, the biological agent and the blood excipient get dissolved in the blood. The dissolution of the biological agent and the blood excipient results in the release of the drug from the two or more nano-carriers at the target site. Thus, a pH dependent release of the drug from the two or more nano-carriers is achieved.

Further, as the encapsulating medium is one or more of the biological agent, the blood excipient and the phospholipid, the two or more nano-carriers exhibit an affinity for the tissues of the target site. Such an affinity facilitates efficient transferring of the two or more nano-carriers from the hydrophilic surface of the inflatable balloon to the target. Further, the one or more of the biological agent and the phospholipid exhibit a property of stabilizing the drug present in the nano-carrier of the two or more nano-carriers.

The two or more nano-carriers may have an average diameter ranging from 10 nm to 1500 nm. Alternatively, two or more nano-carriers may have two or more average diameters. The two or more average diameter may range from 1 nm to 1500 nm.

In accordance with various embodiments, the two or more nano-carriers may be prepared by using the methods known in the art. For example, one or more of the nano-crystals of the drug, the nano-particles of the biological agent, the nano-particles of the phospholipid and the nano-particles of the blood excipient may be obtained by using one or more of, but are not limited to, high-pressure homogenization, spray drying, high-speed homogenization, ball milling, pulverization, sol-gel methods, hydrothermal methods, spray pyrolysis method and the like.

The nano-crystals of the drug are further encapsulated by the nano-particles of one or more of the biological agent, the phospholipid and the blood excipient to obtain the two or more nano-carriers by using methods known in the art. An average diameter of the two or more nano-carriers thus obtained may range from 1 nm to 1500 nm. Subsequently, upon obtaining the two or more nano-carriers, the two or more nano-carriers may be coated on the hydrophilic surface of the drug-delivering insertable medical device using the methods and techniques known in the art.

In an exemplary embodiment, in order to obtain the nano-crystals of the drug, a predetermined amount of the drug may be added into a solvent followed by a surfactant. For example, in order to obtain the nano-crystals of sirolimus, a predetermined amount of sirolimus may be added into water followed by Tween 80. A solution of the drug thus obtained may then be subjected to a high speed homogenization at a predetermined rpm for a predetermined time. The predetermined rpm and the predetermined time may be selected based on a desired average diameter of the nano-crystals of the drug. In response to subjecting the solution of the drug to the high-speed homogenization, at the predetermined rpm and for the predetermined time, a solution of the nano-crystals of the drug is obtained.

Similarly, a predetermined amount of the biological agent, for example, soya phospholipid, may be added into a solvent followed by a surfactant. A solution of the biological agent thus obtained may then be subjected to a high speed homogenization at a predetermined rpm for a predetermined time. The predetermined rpm and the predetermined time may be selected based on a desired average diameter of the nano-particles of the biological agent. In response to subjecting the solution of the biological agent to the high-speed homogenization, at the predetermined rpm and for the predetermined time, a solution of the nano-particles of the biological agent is obtained.

Subsequently, the solution of the nano-crystals of the drug is added to the solution of the nano-particles of the biological agent to form a mixture. The mixture is then subjected to high-speed homogenization at a predetermined rpm for a predetermined time. In response to subjecting the mixture to the high-speed homogenization, at the predetermined rpm and for the predetermined time, the nano-crystals of the drug are encapsulated with the nano-particles of the biological agent to form the two or more nano-carriers. Thus, a solution of the two or more nano-carriers may be obtained.

Thereafter, the solution of the two or more nano-carriers may be extracted with one or more organic solvent (e.g. methanol, dichloromethane, and the like.) to remove any free form of one or more of the biological agent and the drug. A solution resulting from the extraction with one or more organic solvents may then be used to coat the two or more nano-carriers on the hydrophilic surface of the inflatable balloon. The two or more nano-carriers may be coated on the hydrophilic surface of the inflatable balloon by using methods and equipments known in the art. For example, the two or more nano-carriers may be coated on the hydrophilic surface of the inflatable balloon by using one or more of, but are not limited to, a spray coating technique and atomization technique. Further, the two or more nano-carriers may be coated on the hydrophilic surface of the inflatable balloon when the balloon is in a folded configuration and an unfolded configuration.

In an embodiment, the hydrophilic surface of the inflatable balloon is coated with the two or more nano-carriers with an average diameter of 800 nm. The hydrophilic surface further has the one or more exposed hydrophilic surface at a proximal end and a distal end of the hydrophilic surface. In other words, portions at the proximal end and the distal end are not coated with the two or more nano-carriers. A nano-carrier of the two or more nano-carriers includes nano-crystals of sirolimus surrounded by one or more of tricalcium phosphate and soya phospholipid. When the inflatable balloon is inflated upon coming in proximity to the target site in the blood vessel, about 70% to 80% of the two or more nano-carriers are released from the hydrophilic surface of the inflatable balloon. The two or more nano-carriers are then absorbed by tissues at the target site.

In an exemplary embodiment, the inflatable balloon is used for treating the medical condition associated with a blood vessel. The blood vessel may be one of, for example, a coronary artery, a peripheral artery, a carotid artery, a renal artery, an illiac artery, arteries below a knee, and a vein. The blood vessel includes two or more layers of the tissues. The two or more layers of the tissues may be an intima layer, a media layer and an adventitia layer. The intima layer is an innermost layer of tissues of the blood vessel that is in direct contact with the blood flow through the blood vessel. The media layer is a layer of tissues of the blood vessel that is beneath the intima layer. Whereas, the adventitia layer is a layer of tissues of the blood vessel that is beneath the media layer.

Upon being released from the hydrophilic surface of the inflatable balloon, the nano-carrier of the two or more nano-carriers may penetrate the intima layer directly through inter-tissue pores present in the intima layer. Whereas, the nano-carrier of the two or more nano-carriers may penetrate the media layer by passing through the inter-tissue pores present in the intima layer and a vasa vasorum associated with the media layer. Similarly, the nano-carrier of the two or more nano-carriers may penetrate the adventitia layer by passing through the inter-tissue pores present in the intima layer, the vasa vasorum associated with the media layer and a vasa vasorum associated with the adventitia layer. The inter-tissue pores present in the intima layer, the vasa vasorum associated with the media layer and the vasa vasorum associated with the adventitia layer have different internal diameters. Therefore, penetration of the nano-carrier of the two or more nano-carriers into one or more of the intima layer, the media layer and the adventitia layer depends upon an average diameter associated with the two or more nano-carriers.

In another embodiment, the two or more nano-carriers include, a first set of nano-carriers, a second set of nano-carriers, and a third set of nano-carriers. The first set of nano-carriers has a first average diameter suitable for penetrating the intima layer through the inter-tissue pores present in the intima layer. The second set of nano-carriers have a second average diameter suitable for penetrating the media layer through the vasa vasorum associated with the media layer and the inter-tissue pores present in the intima layer. The third set of nano-carriers have a third diameter suitable for penetrating the adventitia layer through the inter-tissue pores present in the intima layer, the vasa vasorum associated with the media layer and the vasa vasorum associated with the adventitia layer.

The first average diameter may range from 800 nm to 1500 nm, the second average diameter may range from 300 nm to 800 nm and the third average diameter may range from 10 nm to 300 nm. In an embodiment, the first average diameter is 1000 nm, the second average diameter is 700 nm and the third average diameter is 200 nm. The first average diameter, the second average diameter and the third average diameter may be varied to meet a particular therapeutic need without departing from the scope of the invention.

The first set of nano-carriers with the first average diameter may include about 10% to 60% of the two or more nano-carriers. Whereas, the second set of nano-carriers with the second average diameter may include about 20% to 70% of the two or more nano-carriers and the third set of nano-carriers with the third average diameter may include about 30% to 80% of the two or more nano-carriers. Alternatively, the first set of nano-carriers, the second set of nano-carriers and the third set of nano-carriers may include about 15% to 90%, 10% to 85%, and 5% to 85% of the two or more nano-carriers, respectively.

When the two or more nano-carriers are released from the hydrophilic surface of the inflatable balloon, the first set of nano-carriers penetrate the media layer through the inter-tissue pores present in intima layer. The second set of nano-carriers penetrate the media layer through the vasa vasorum associated with the media layer and the inter-tissue pores present in the intima layer. Whereas, the third set of nano-carriers penetrate the adventitia layer through the vasa vasorum associated with the adventitia layer, the vasa vasorum associated with the media layer and the inter-tissue pores present in the intima layer. Thus, a size dependent penetration of the two or more nano-carriers is achieved.

In yet another embodiment, the drug present in the third set of nano-carriers may be different from a drug present in the second set of nano-carriers and the drug present in the second set of nano-carriers may be different from a drug present in the first set of nano-carriers. In other words, the drugs present in each of the first set of nano-carriers, the second set of nano-carriers and the third set of nano-carriers may be different. For example, the drug present in the third set of nano-carriers may be one or more of an anti-inflammatory agent and an anti-thrombogenic agent. The second set of nano-carriers may include an anti-proliferative agent. Whereas, the first set of the nano-carriers may include, for example, a pro-healing agent. Further, the drug present in the one or more of the first set of the nano-carriers, the second set of the nano-carriers and the third set of nano-carriers may be selected from one or more of, but not are limited to, an anti-neoplastic agent, an anti-coagulant agent, an anti-fibrin agent, an antithrombotic agent, an anti-mitotic agent, an antibiotic agent, an anti-allergic agent and an antioxidant, estrogens, a protease inhibitor, antibodies, an immunosuppressive agent, a cytostatic agent, a cytotoxic agent, a calcium channel blocker, a phosphodiesterase inhibitor, a prostaglandin inhibitor, a dietary supplement, vitamins, anti-platelet aggregating agent and genetically engineered epithelial cells without departing from the scope of the invention.

The third set of nano-carriers have a smallest average diameter among the first set of nano-carriers, the second set of nano-carriers and the third set of the nano-carriers. Therefore, the time required for the third set of nano-carriers to release from the hydrophilic surface upon coming in proximity of the target site is less than the time required for the second set of nano-carriers and the first set of nano-carriers to release from the outer surface. Thus, the third set of nano-carriers exhibit a rapid rate of release from the hydrophilic surface. Whereas, the second set of nano-carriers and the third set of nano-carriers exhibit slower rates of release from the hydrophilic surface as compared with the rate of release of the third set of nano-carriers.

Additionally, because of the presence of one or more of the biological agent, the phospholipid, and the blood excipient in the two or more nano-carriers, the two or more nano-carriers exhibit an affinity for the tissues of the target site. Further, owing to the capability of the two or more nano-carriers to penetrate the one or more layers of the blood vessel, nano-carriers with a smallest average diameter penetrate up to the adventitia layer. The nano-carriers that penetrate into the adventitia may remain in the adventitia layer for a prolonged time. In other words, the adventitia layer may act as a reservoir of the drug from where the drug is slowly released over the prolonged time.

The drug present in the nano-carrier of the two or more nano-carriers is released only after the nano-carriers penetrates one or more of the intima layer, the media layer and the adventitia layer and after the dissolution of the encapsulating medium. Thus, an in-tissue release of the drug at the target site is achieved. The drug thus released may diffuse across one or more of the adventitia layer, the media layer and the intima layer during the prolonged time. In such an instance, the drug that is diffused across the one or more of the adventitia layer, the media layer and the intima layer may provide an in-tissue diffusion of the drug for the prolonged time. Thus, because of the in-tissue release of the drug and the in-tissue diffusion of the drug, the drug may be delivered to maximum portion of a lesion at the target site.

In still yet another embodiment, the inflatable balloon is used to deliver the drug to a target site where a stent is deployed in the blood vessel. In such an instance, owing to the in-tissue release and in-tissue penetration of the drug for the prolonged time, the instances of delayed healing of lesions at the target site and improper healing of the lesions at the target site are minimized. Thus, an anti-platelet therapy that has to be given to the patients with delayed healing or improper healing of the lesions may be minimized.

In accordance with various embodiments, the two or more nano-carriers may be coated on the hydrophilic surface of the inflatable balloon in any manner so as to achieve a particular therapeutic objective without departing from the scope of the invention. The manners in which the two or more nano-carriers may be coated on the hydrophilic surface includes, one or more of, but are not limited to, coating the two or more nano-carriers as a single layer on the hydrophilic surface, coating the two or more nano-carriers as two or more layers on the hydrophilic surface and coating the two or more nano-carriers using one or more polymers. Further, the two or more nano-carriers may be coated on the hydrophilic surface of the inflatable balloon by using one or more techniques, for example, but are not limited to, spray-coating, deep-coating, ultrasonic cloud coating, mist coating, electrostatic coating and any other technique known in the art.

Generally speaking, pursuant to various embodiments, the invention also discloses a method for delivering the drug to the target site in a body lumen for treating the medical condition associated with the body lumen. The method includes positioning the inflatable balloon at the target site in the body lumen. The inflatable balloon may be positioned at the target site by using the balloon catheter assembly and the methods known in the art. After the inflatable balloon is positioned at the target site, the inflatable balloon is inflated using suitable inflating mechanism associated with the balloon catheter assembly. In response to the inflating, the inflatable balloon comes in contact with the target site and about 30% to 80% of the two or more nano-carriers are released from the inflatable balloon within 15 to 90 seconds upon contacting the target site. In accordance with various embodiments, the inflatable balloon may be brought into the contact with the target site for one or more times to deliver the drug to the target site.

### Example 1

Soya phospholipid was obtained from Lipoid GMBH, Batch No.: 776114-1/906. Sirolimus was obtained from Fujan Chemicals, China with purity greater than 99.5%. The water, other solvents and reagents used were of HPLC grade. A polyamide catheter system with COPAN Co-Polyamide angioplasty balloon (herein after “the balloon system”) coated with Hydrophilic coating (hereinafter “the hydrophilic surface”) was obtained from Minvasys, Paris, France.

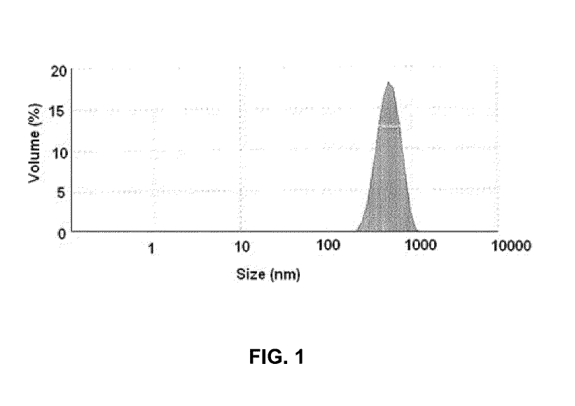

Soya phospholipid (20 mg w/w) was added to HPLC grade water (20 ml) followed by Tween 80 (5 mg) to obtain aqueous solution of soya phospholipid. The aqueous solution of soya phospholipid (20 ml) was subjected to a high speed homogenization at 15000-20000 rpm for 20 to 25 minutes in ice-cold water bath to obtain Solution A1. The Solution A1 thus obtained contained nano-particles of soya phospholipid. The solution A1 was subsequently analyzed for particle size detection using Malvern ZS90 (Malvern, UK) size detector. FIG. 1 illustrates the size distribution of nano-particles of soya phospholipid as detected by Malvern Z590. The average diameter of the nano-particles of the soya phospholipid was found to be 431 nm with largest diameter up to 1000 nm.

Sirolimus (20 mg w/w) was added to 10 ml of de-ionized water to obtain aqueous solution of sirolimus. The aqueous solution of sirolimus (10 ml) was subjected to a high speed homogenization at 15000-20000 rpm for 150 to 200 minutes in ice-cold water bath to obtain Solution A2. The Solution A2 thus obtained contained nano-crystals of sirolimus.

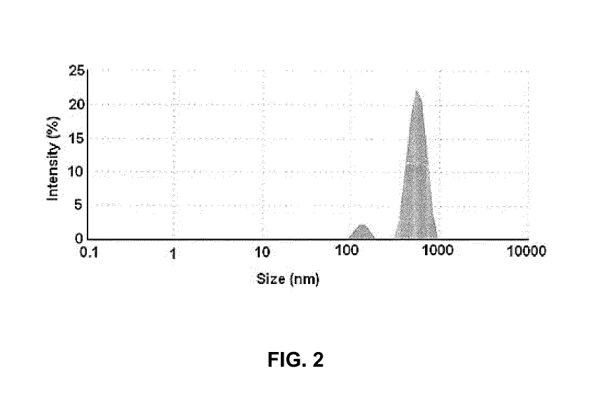

Solution A1 was immediately added to Solution A2 drop by drop slowly under high speed homogenization process. The resultant mixture was subjected to a high speed homogenization at 15000-20000 rpm for another 20 minutes after complete addition to obtain Solution A3. Solution A3 was then stirred with a magnetic stirrer (2MLH hot plate heater cum stirrer, Accumax, INDIA) for 20 minutes. Solution A3 thus obtained contained nano-carriers (nano-crystals of sirolimus surrounded by nano-particles of soya phospholipid). Solution A3 was subsequently analyzed for particle size detection using Malvern ZS90 (Malvern, UK) size detector. FIG. 2 illustrates the size distribution of nano-carriers as detected by Malvern Z590. The average diameters of nano-carriers were found to be 133.6 nm (Peak 1) and 554.9 nm (Peak 2) with maximum diameter being up to 1000 nm.

Solution A3 (Aqueous solution of nano-carriers) was further subjected to extraction with dichloromethane. Solution A3 (20 ml) was transferred to 100 ml separating funnel. 50 ml of dichloromethane was added to the 100 ml separating funnel. The resultant mixture was shaken for 15 min and then allowed to stand. Thereafter, two layers i.e. aqueous layer and the dichloromethane layer were observed in the 100 ml separating funnel. The dichloromethane layer was separated from the aqueous layer. The dichloromethane layer i.e. solution of the nano-carriers was stored in amber colored small measuring flask with batch number. Subsequently, the solution of the nano-carriers was used for coating the balloon system.

The solution of the nano-carriers (5 ml) was fed into the reservoir of a coating machine. The balloon system was mounted on a rotating mandrel of the coating machine. The balloon of the balloon system was exposed to the atomization nozzle of the coating machine. The balloon system was rotated at 5 to 40 rpm by rotating the mandrel and simultaneously the solution of nano-carriers was sprayed over the balloon at 0.5-4.0 psi inert gas pressure and 2 oscillations. Thus, the balloon coated with the nano-carriers (hereinafter “the coated balloon”) was obtained. The coated balloon system was then removed and checked under high resolution microscope for the coating surface smoothness and any foreign particles. The coated balloon system was then subjected to further analysis as explained in Example 2 below.

### Example 2

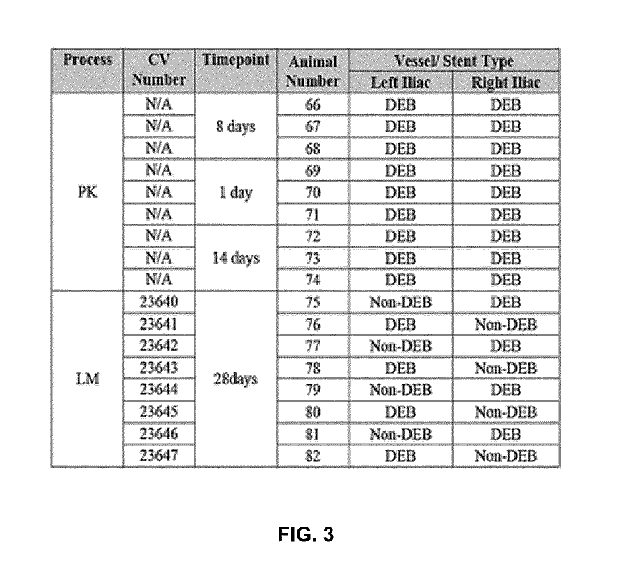

The coated balloon was further evaluated in-vivo in 17 male New Zealand rabbits, 5 to 6 months old and weighing between 3 and 4 Kg (hereinafter “animals”) for PharmacoKinetic (PK) study and histological evaluation or Light Microscopy (LM). FIG. 3 is a table illustrating numbers assigned to 17 animals, stent type and location of stent in each animal and the type of study (PK/LM) conducted on the animals. Out of the 17 animals 9 (Animals 66 to 74) were used for PK study and 8 (Animals 75 to 82) were used for histological evaluation.

The coated balloons were inserted into both the iliofemoral arteries of the animals used for the PK study. The coated balloons were inflated twice in the iliofemoral arteries. The coated balloons were first inflated for 70 seconds at 7 ATM and then deflated. Again, the coated balloons were inflated for the second time for 60 seconds at 7 ATM and then deflated and withdrawn.

A pre-mounted stent (3.0 mm× 12-14 mm) i.e. a stent mounted on the coated balloon was implanted in the right and the left iliac artery of the animals at a nominal pressure with 30 seconds of balloon inflation. The balloon to stent ratio for all the pre-crimped stents was approximately 1.4:1. Following the deployment of the stents, angiography was performed to examine the patency of the stents.

Whole blood was collected from the central ear of the animals for serum analysis at four time points, namely 0.5 Hour, 1 Hour, 3 Hour and 24 Hour post deployment of the stents. After euthanasia at the respective time point, the tissue around the stent was dissected free, weighed, and snapfrozen in liquid nitrogen for later measurement. The University of Colorado Denver performed drug analysis of whole blood and the tissue surrounding the stent. The concentration of sirolimus at the four time points i.e. 0.5 Hour, 1 Hour, 3 Hour and 24 Hour was found to be 9.32 ng/ml, 7.08 ng/ml, 4.09 ng/ml and 0.81 ng/ml, respectively.

It was concluded that at Day 1, Day 8, and Day 14 pharmacokinetics study, maximal blood concentrations of sirolimus were seen at 30 minutes post catheterization (9.3 ng/ml) while circulating levels decreased markedly by 24 hours (0.81 ng/ml). For tissue drug levels, maximal concentrations were achieved at Day 1 (140.4 ng/mg), which showed a significant decreased to 15.5 ng/mg by Day 8 and 5.5 ng/mg by Day 14. Individual drug concentrations at each time point however, show variation among arteries where values at Day 1 ranged from 35.0 to 275.0 ng/mg, values at Day 8 ranged from 0.7 ng/mg to 33.2 ng/mg and values at Day 14 ranged from 14.8 ng/mg to below limits of quantification [BLQ] in two animals (Animal number 72 and 74). In comparison, published data in the rabbit with a sirolimus-eluting stent at similar time points achieved tissue concentrations of 4.52 ng/mg and 1.56 ng/mg at Day 1 and Day 8, respectively (Finn A V, Kolodgie F D Circulation 2005; 112:270).

Further measurements included histological study of the slides of the tissues around the stents. The slides were analyzed by using National Institute for Standard and Technology calibrated microscope system (IP Lab Software, MD). The cross-sectional areas for each slide i.e. External Elastic Lamina (EEL), Internal Elastic Lamina (IEL) and the Lumen Area were measured. Neointimal thickness was measured as a distance between the inner surface of the stent strut and the Luminal border of the stent. Vessel layer areas were calculated using the following formulae:

Area of Media=EEL−IEL,

Neointima Area=IEL−the Lumen Area and

Percent Stenosis=[1−(Luminal Area/IEL)]*100.

All the stents were found to be widely expanded and well apposed to the vessel walls. All the animals survived the in-life-phase of the study.

Re-endothelialization was found to be complete for the sample group and the control group. Percent Stenosis was found to be 11.48 (±1.30%) in sample group and 11.49 (+1.49%) in the control group. The Neointimal Thickness was found to be 0.030 mm (±0.0076 mm) in sample group and 0.032 (+0.0098 mm) in the control group.

In-tissue sirolimus concentration was also evaluated at Day 1, Day 8 and Day 14. The in-tissue sirolimus concentration at Day 1, Day 8 and Day 14 were found to be 140.4 ng/mg, 15.5 ng/mg and 5.5 ng/mg respectively. It was concluded that relatively higher in-tissue concentrations can be achieved with the coated balloon with single inflation as compared with the published data of study in rabbits pertaining a sirolimus-eluting stent with similar time points (Finn A V, Kolodgie F D Circulation 2005; 112:270).

### Example 3

6 Brazilian pigs (Hereinafter “the animals”) weighing about 25 to 30 kg were selected for the study. Bare metal stent (Cronus®, obtained from, Scitech, Brazil), with sizes of stent ranging from 2.5*13 mm to 3.0*13 mm and the coated balloons with sizes of about 3.0*15 mm were used. The stents were deployed in three vessels i.e. LAD (Left Interior Descending), LCX (Left Circumflex) and RCA (Right Coronary Artery of each animal by using: a) the coated balloon (Sirolimus), b) the coated balloon (Paclitaxel) and c) a bare balloon. The Paclitaxel coated balloon was prepared by replacing sirolimus in example 1 with Paclitaxel and rest of the process was kept same. The balloon to artery ratio for each coated balloon was approximately 1.1:1.0 to 1.2:1.0. Each of the coated balloon and the bare balloon were inflated for 60 seconds. The animals with sirolimus coated balloon were labeled “Sirolimus”, the animals with Paclitaxel coated balloon were labeled “Paclitaxel” and the animals with bare balloon were labeled “Control”.

The groups i.e Sirolimus, Paclitaxel and Control were each studied for qualitative OCT analysis. The qualitative parameters that were studied included: a) Tissue Proliferation at the stent edges, b) Intra-luminal Thrombus Formation and c) Uncovered Stent Struts. It was found that the Tissue Proliferation at the stent edges was found in one Control and two Paclitaxel. Whereas, no incidence of Tissue Proliferation at the stent edges was observed in the Sirolimus group. Further, it was found that the Intra-luminal Thrombus Formation was found in one Control and two Paclitaxel. Whereas, no incidences of Intra-luminal Thrombus Formation were observed in the Sirolimus group.

It was concluded that sirolimus performed better than paclitaxel and bare balloon in terms of reduction in neointimal growth and endothelialization. The stent strut coverage by tissue was virtually complete for all the groups. Toxic effect was found in paclitaxel group. The toxic effect was found in the paclitaxel group due to greater in-tissue concentration of paclitaxel. However, the dose of the paclitaxel used was lower as compare with commercially available products. Thus, the coated balloons showed acute transfer of paclitaxel or sirolimus using a polymer free approach.

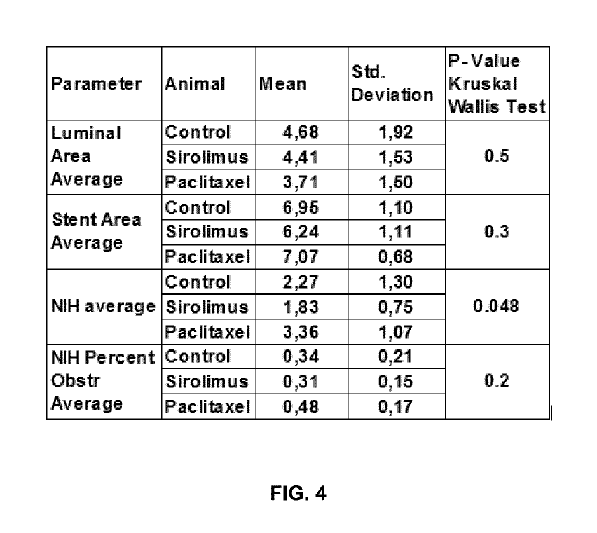

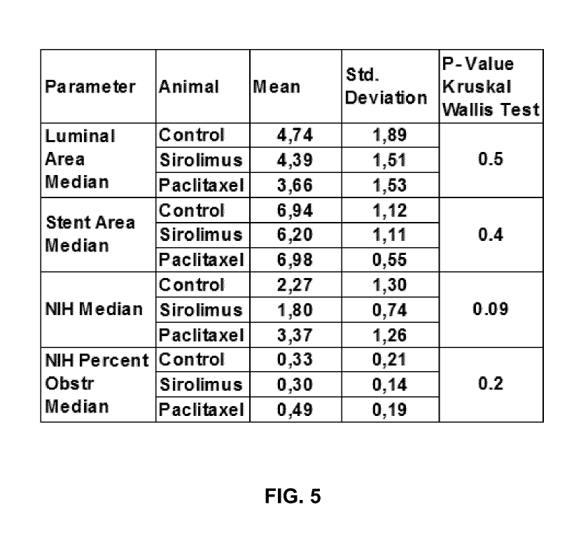

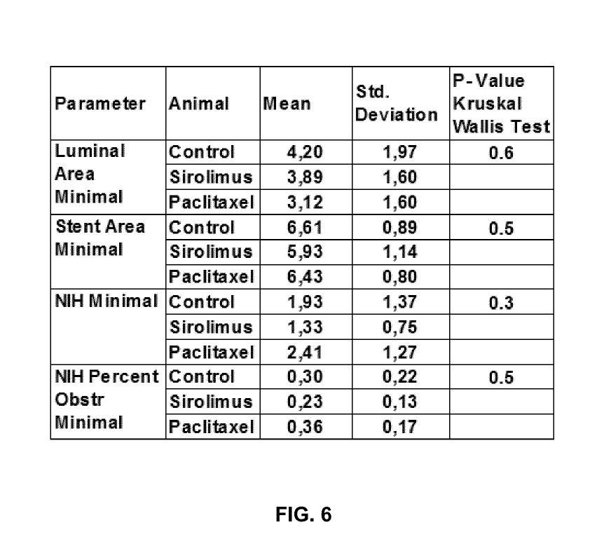

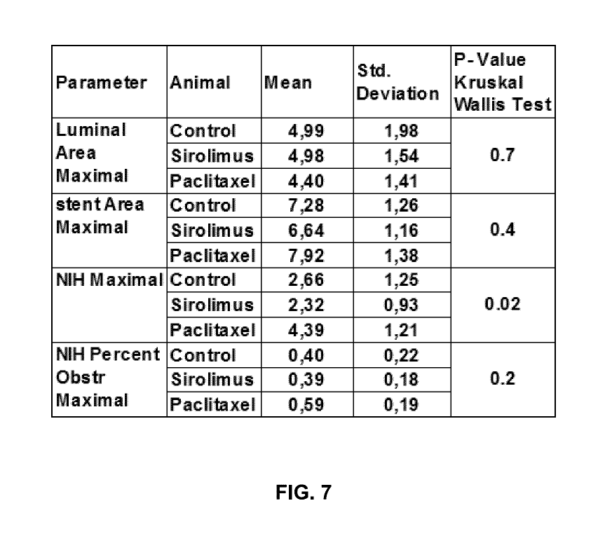

Quantitative OCT analysis was performed on six equidistant in-stent tissue slices for each stent. Thus a total of 108 [i.e. 6 (animals)*3 (arteries each)*6 (tissue slices)] in-stent tissue slices (cross-sections) were analyzed. The parameters for quantitative analysis included: a) Lumen Area, b) Stent Area, c) Neointimal Area and d) Percent Neointimal Obstruction. FIG. 4 illustrates a table of values of mean and standard derivations of the Average Luminal Areas, Average Stent Areas, Average Neointimal Areas and Average Percent Neointimal Obstruction findings for each group i.e. Control, Sirolimus and Paclitaxel. FIG. 5 illustrates a table of values of mean and standard derivations of the Median Luminal Areas, Median Stent Areas, Median Neointimal Areas and Median Percent Neointimal Obstruction findings for each group. FIG. 6 illustrates a table of values of mean and standard derivations of the Minimal Luminal Areas, Minimal Stent Areas, Minimal Neointimal Areas and Minimal Percent Neointimal Obstruction findings for each group. FIG. 7 illustrates a table of values of mean and standard derivations of the Maximal Luminal Areas, Maximal Stent Areas, Maximal Neointimal Areas and Maximal Percent Neointimal Obstruction findings for each group. It was concluded that out of a data set of the 927 stent struts, just one strut (Paclitaxel) was found to be unequivocally uncovered by the tissues. Based on observation of thrombus it was concluded that drug dose of paclitaxel might be higher than required. The higher dose and vis-a-vis the thrombus effect of the paclitaxel may be attributed to higher in-tissue drug penetration of paclitaxel. Thus the amount of sirolimus of paclitaxel to be coated on the coated balloon is lesser than the amount of sirolimus of paclitaxel loaded on current DEBs.

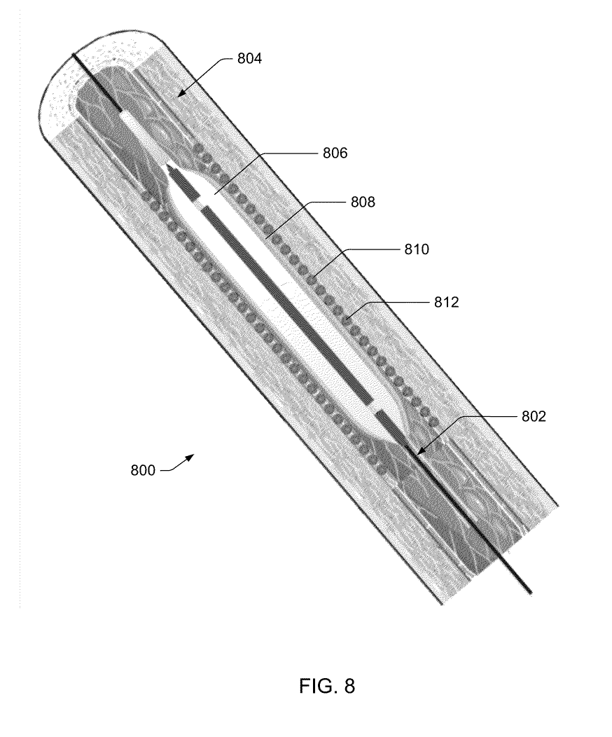

FIG. 8 is a diagram of a non-implantable drug-delivering medical device 800 on a catheter 802, shown in a lumen of a vessel 804, that includes (for example) a balloon 806, inflatably positioned on the catheter 802, the inflatable balloon 806 having a hydrophilic layer 808, at least one portion of the hydrophilic layer 808 being coated with a plurality of nano-carriers 810 (nano-carriers not shown to scale), with at least one type of the nano-carriers 810 surrounded by an encapsulating medium 812.

### Example 4

### Method: In-Vivo PK and Histology Study

Experiments were performed in a total of 17 (9 for PK and 8 for histopathology) New Zealand white male rabbits, 5-6 Months, 3.0-4.0 kg. Through the left common carotid artery, under fluoroscopy, both iliofemoral arteries were injured by endothelial denudation with a 3F Fogarty embolectomy catheter. Immediately after the arterial balloon denudation, also under fluoroscopy, a DCB was inflated for 60 seconds (7 atm) in both iliac arteries, followed by bare metal stents were implanted in each iliac artery over the same site, under fluoroscopy. Nine animals (n=18) were euthanized at 1, 8, and 14 days PK estimation (390 mg sodium pentobarbital and 50 mg phenytoin sodium) and tissue around the stent was dissected free, weighed, and snap-frozen in liquid nitrogen for drug measurement by HPLC-tandem mass spectrometry. Eight animals were analysed for histopathology at 28 days.

Results

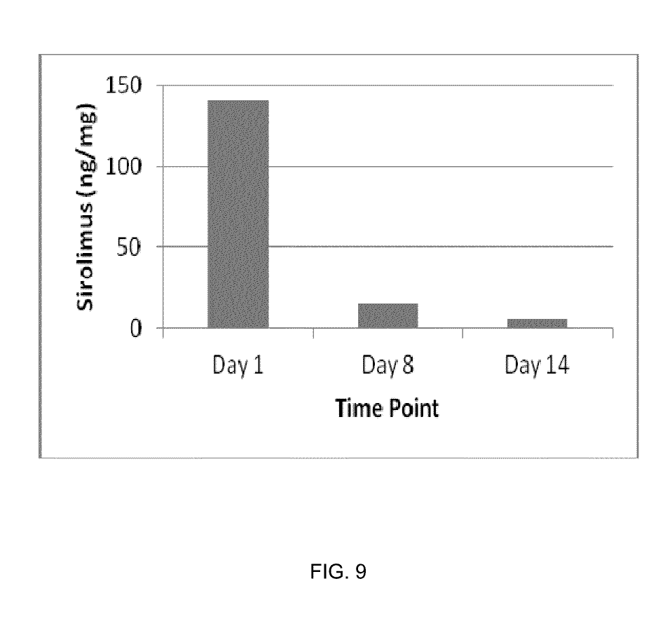

FIG. 9 shows the pk data of sirolimus coated balloon. In-tissue uptake of sirolimus was measured at 1, 7 and 14 day was 140.6 ng/mg, 15.5 ng/mg and 5.5 ng/mg respectively. The highest amount was sirolimus was 275.6 ng/mg, 33.2 ng/mg and 14.8 ng/mg at day 1, 8 and 14 respectively.

### Example 5

### Method: In-Vivo Multiple Inflation PK Study

For toxic level finding using pharmacokinetic studies, PCI with DCB (single and 3× inflations) was performed in bilateral iliac arteries (n=8 treatment sites) at 8 days. Immediately following bilateral iliac artery balloon denudation, DCB was inflated at its nominal inflation pressure for 60 seconds to treat the injured iliac artery. Following the PCI, the balloons were retrieved and the amount of drug remaining on balloon was calculated. The vessels were harvested at 8 days, snap-frozen, and later analyzed for arterial sirolimus. The residual drug remain on the device was also measured.

Results: In-Vivo Multiple Inflation PK Study and Residual Drug on Balloon

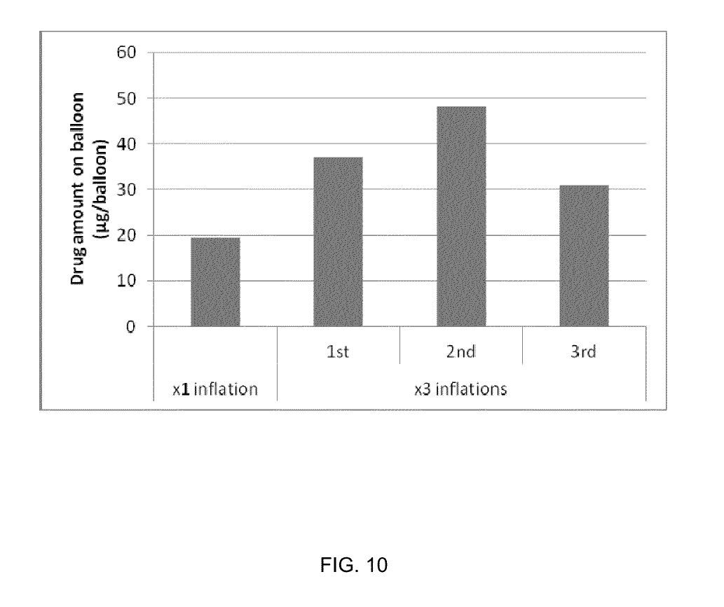

Drug amounts remaining on the balloons following treatment were variable with a range of 4.68 μg/balloon (minimum) to 60.90 μg/balloon (maximum). Inflation time (×1 or ×3: 1^{st}, 2^{nd}, or 3^{rd}) demonstrated a trend towards lower values in the xl inflation group, as in FIG. 10.

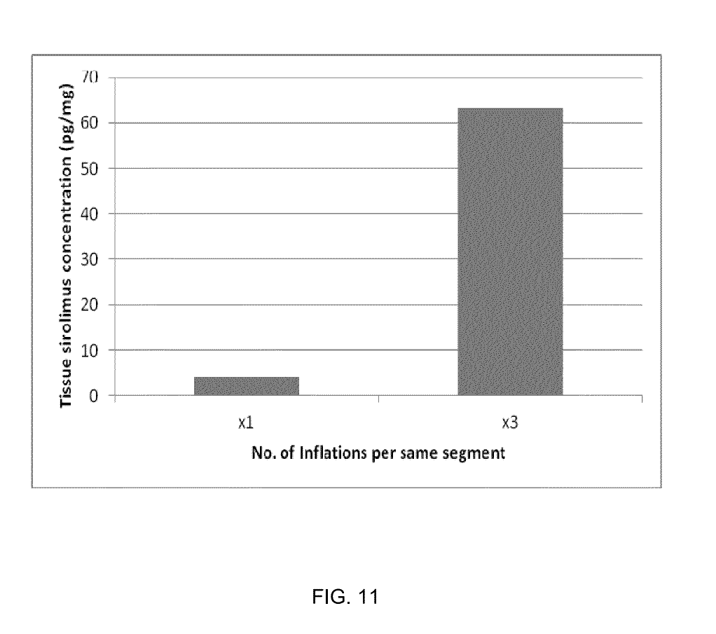

Drug concentration of vessel at 8 days following DCB treatment was lower in ×1 inflation group than in ×3 inflation group as expected, as in FIG. 11. In the ×1 inflation group, the values converged within a range of 3.58 pg/mg (minimum) to 9.77 pg/mg (maximum) while the ×3 inflation group demonstrated a large divergence with a maximum value of 13566.78 pg/ml.

### Example 6

### Confocal Study to Assess SRL Distribution in Arterial Layers Over Time, Showing how Drug Penetrates to Different Layer of Artery in Non-Significant Lesions

The study was performed in New Zealand white rabbits at 1 hr, 24 hr, 3, and 7 days time point. Ilio-femoral arteries (n=8) were treated bilaterally with DTF-SRL. The samples were inflated at 6 ATM for 60 second. The samples were harvested and imaged longitudinally “en face” and in histologic cross-sections by confocal microscopy.

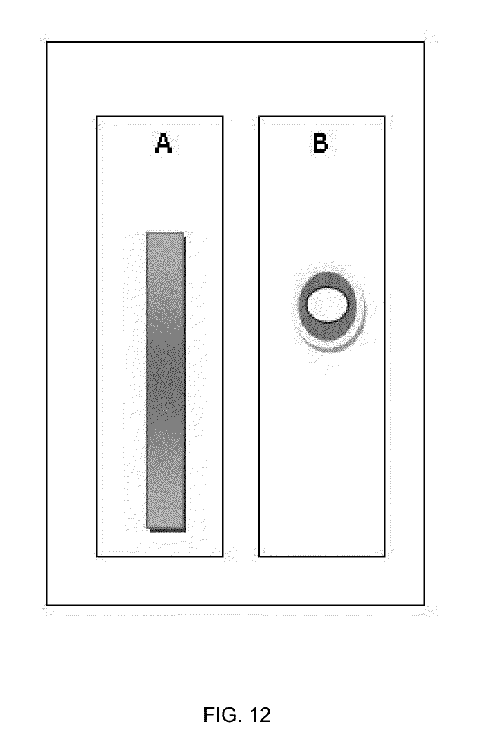

To determine the extent and distribution of nano particle sirolimus on the luminal surface, ilio-femoral arteries were opened longitudinally and positioned face down on a histologic slide in aqueous mounting media, as in FIG. 12, strip “A”. The DTF-labeled nanoparticle sirolimus adherent to the luminal surface of the artery was viewed en face using a Zeiss Pascal confocal microscopy, equipped with a 488-nm excitation argon laser (green channel) where images were acquired under a ×40-oil immersion objective.

Frozen Sectioning for Confocal Microscopy

The depth and circumferential distribution of DTF-labeled nanoparticle sirolimus was also examined in histologic sections prepared in cross-section. For these studies, 2 to 3-mm unfixed artery segments were snap-frozen in liquid nitrogen cooled isopentane and serial cut at 10 μm using a standard cryostat (Thermo Shandon Cryotome® e). Histologic sections were mounted on glass slides, as in FIG. 12, slide “B”, and similarly view by confocal microscopy.

Confocal Study to Assess SRL Distribution in Arterial Layers Over Time

Residual DTF-nSRL Remaining on the Balloon after Artery Treatment

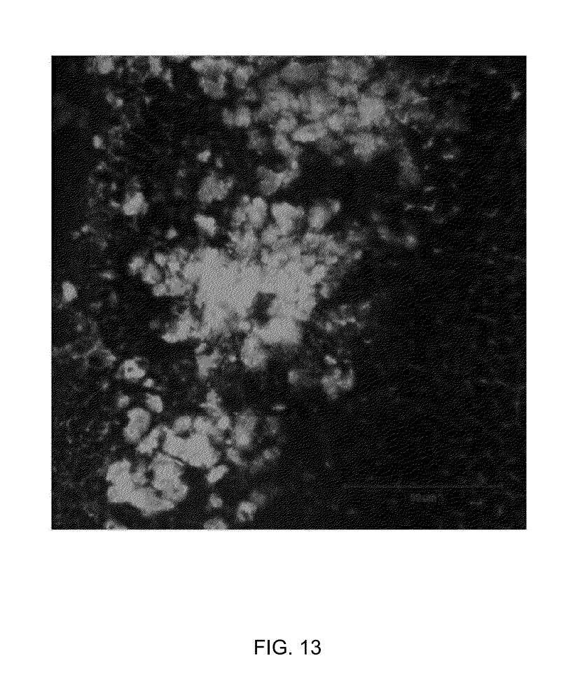

The remaining amount of DTF-nSRL (n=2) was viewed under a confocal microscopy. The representative distributions of remaining DTF-nSRL are seen as solid and weakly diffuse patches of green fluorescence, as in FIG. 13. In general, 30 to 40% of the balloon surface was visually covered by the DTF-label.

1 Hour Drug Distribution

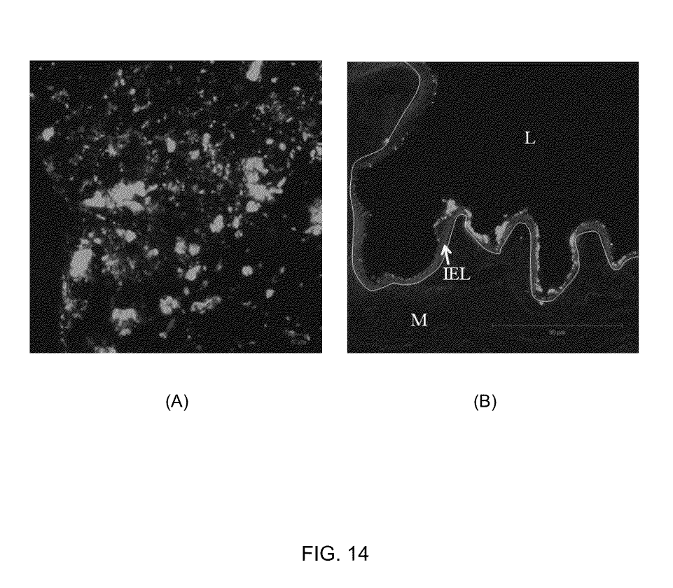

In the 1 hr en face sample shown in FIG. 14, image A, a 5-10% area was observed with strong distribution area of DTF-nSRL signals while 25-35% showed a more diffuse distribution. In histologic sections, the DTF-nSRL was mostly confined to the luminal surface with 60-70% coverage of the circumferential area. Virtually no DTF signal was seen below the level of the internal elastic lamina (IEL) at 1 hr. A solid to diffuse bright green fluorescence (DTF-label) is noted on the IEL surface, as in FIG. 14, image B.

24 Hour Drug Distribution

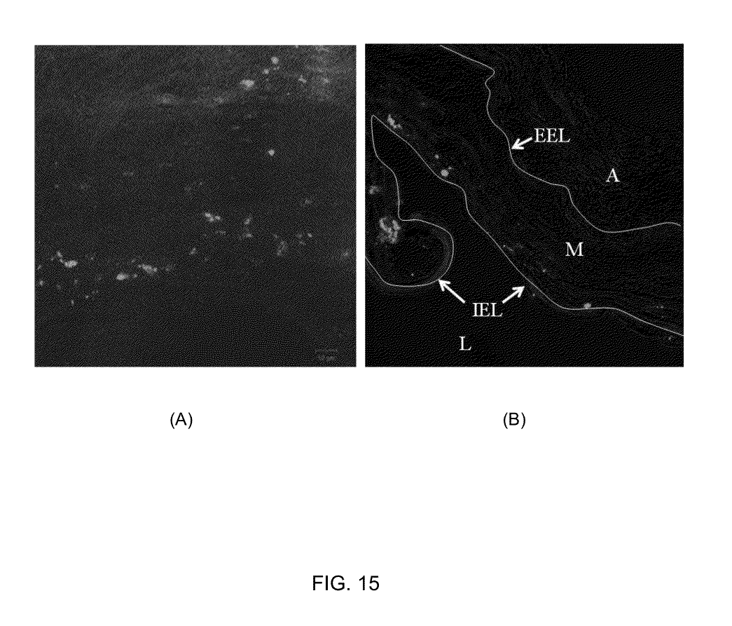

In the en face sample, strong to moderate signals were detected over approximately 5% of the treated area while less moderate to weaker diffuse signals were found in approximately 25-35% of the surface, as in FIG. 15, image A. Histological cross-sections showed that DTF-nSRL was located near the luminal surface in approximately 30-40% of the circumferential area. Overall, the majority of the DTF signal was observed at or below the IEL area, as in FIG. 15, image B.

3 Day Drug Distribution

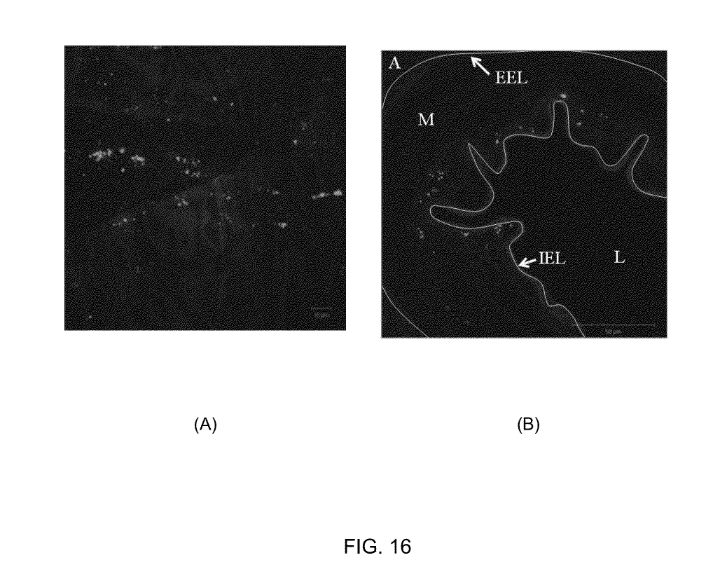

In the en face sample, shown in FIG. 16, image A, moderate to strong signal intensity was visualized in approximately 3-5% of the treated surface while less moderate to low fluorescence was observed in approximately 20-30% of the surface area. In the histologic cross-sections, DTF-nSRL could be observed on the luminal surface involving approximately 30-40% of the circumferential area, as in FIG. 16, image B. The majority of DTF signal below the IEL surface with some positive signals deeper within the medial region.

7 Day Drug Distribution

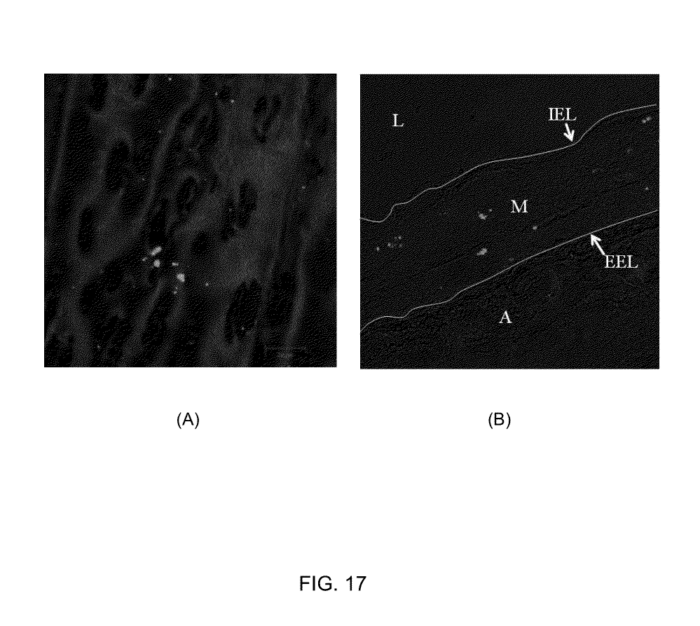

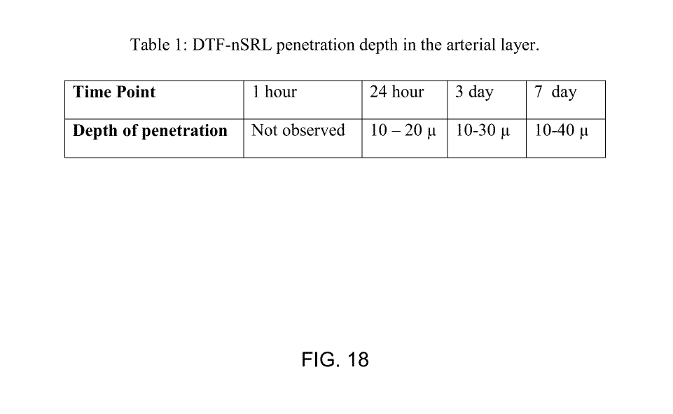

In the en face sample, shown in FIG. 17, image A, low signal DTF signal strength could be visualized in approximately 5% of the treated surface while more diffuse very weak fluorescence was observed in approximately 20-25% of the surface area. Histological sections showed DTF-nSRL near the luminal surface involving approximately 30-40% of the circumferential area, as in FIG. 17, image B. The signal was primarily observed in the medial layer, with majority of the signals deep within the media with rare extension into the adventitial layer. In overall results the drug was consistently travelling from luminal to adventitial direction as shown in FIG. 18.

Coating Surface Characterization:

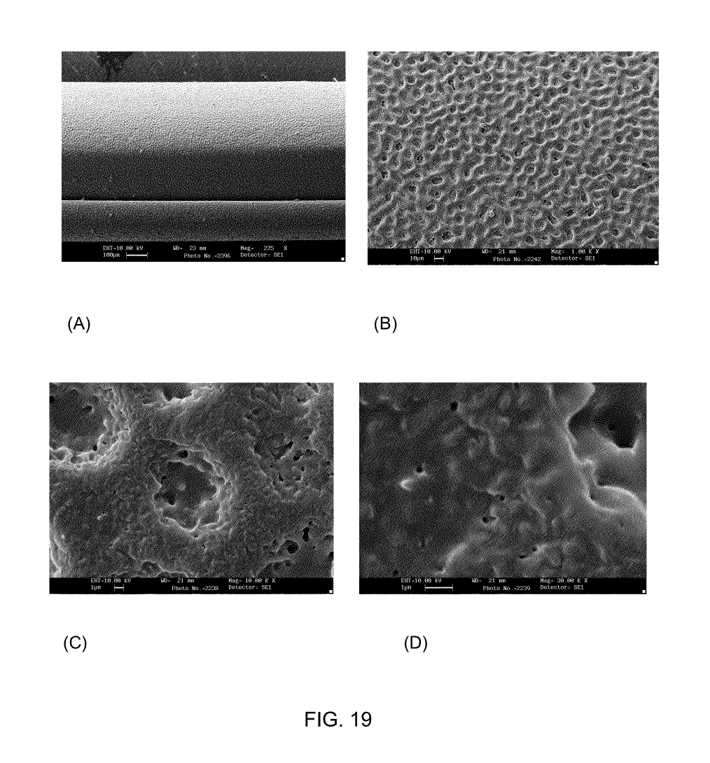

High resolution SEM, as shown in FIG. 19, imaged A-D, revealed that the surface was smooth without defect and irregularities, cracking or deformation. Magnification up to 30KX shows homogeneous nanoparticles distribution of sirolimus throughout the balloon surface. This image further supports presence of sirolimus of coated device.

Characterization of SRL NP and In-Vitro Analysis

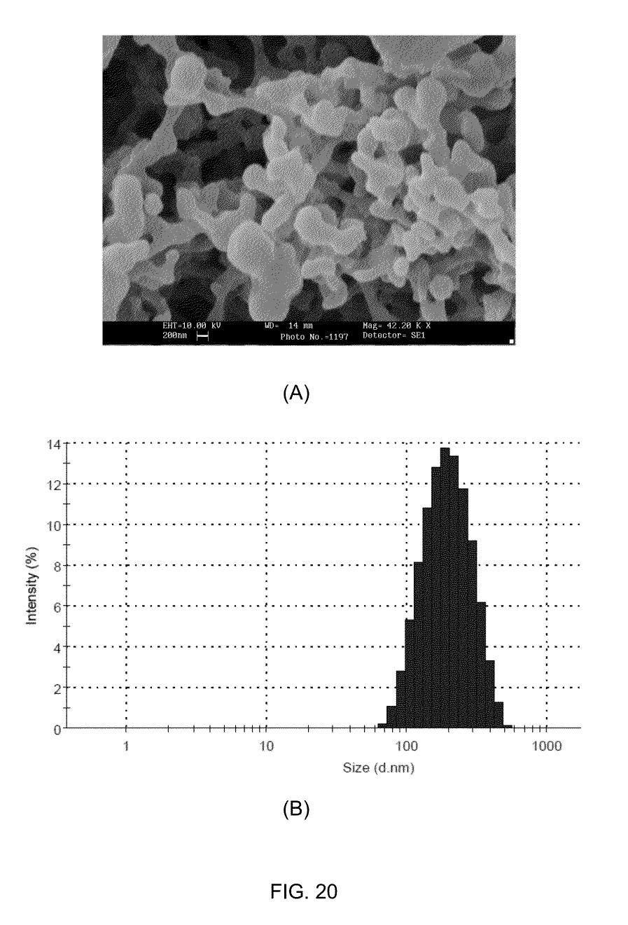

Z-average particle size diameter was <400 nm and zeta potential was −33 mV which revealed moderate stable solution. FIG. 20, image A, shows a particle size analysis, and FIG. 20, image B, shows SEM of sirolimus nanoparticles. The sirolimus content per 3.0*15 mm balloon in folded geometry was average 184+11 μg (n=10). In-vitro release was 56% of total load on first inflation and 26% remaining on the device with 18% remain on the balloon.

In an implementation, a drug delivery device or instrument carries one or more drugs, such as drugs encased as the above-described nano-carriers, to target non-significant lesions in human blood vessel, organ cavity, sac, capsule, lining, layer, coating, membrane, connective tissue, fluid surrounding an organ, and so forth.

At present, there are around 40% of cases in which the patient has a non-significant lesion, wherein the lesion is either treated with POBA: “plain old balloon angioplasty” in which the lumen stenosis of an artery is treated by balloon dilatation only, without applying a stent, or else the non-significant lesion remains untreated.

Though body physiology, the non-significant lesion often becomes significant over a period of time. Then, the patient may need a clogged artery to be opened with a stent and/or a balloon coated with a drug, or even with a non-coated balloon or instrument.

If such a patient is treated by drug eluting balloon (DEB) at the time of initial detection of the non-significant lesion, the early treatment could avoid a future emergency situation, and save many lives.

In an implementation, a non-implantable medical device coated with nano-carriers treats the non-significant lesion by eluting a drug to reduce the non-significant lesion.

A lesion, area of inflammation, disease state, and the like may exist in human tissue layers enclosing an organ, or in the organ itself. A non-implantable device bearing one or more drugs as nano-carriers can deliver the drugs into the one or more tissue layers or organ. The tissue layers enclosing an organ may take the form of a blood vessel (i.e., blood as the organ), organ cavity, sac, capsule, lining, layer, coating, membrane, connective tissue, fluid surrounding an organ, and so forth.

In an implementation, a non-implantable medical device delivers one or more drugs into the pericardial space of a patient to prevent atherosclerosis progression, wherein the one or more drugs can be in the form of nanoparticles, stealth liposome, or phospholipids.

In the same or another implementation, the one or more drugs in the form of nanoparticles, stealth liposome, or phospholipids are injected into the pericardial space or other body space or part.

In an implementation, a non-implantable medical device delivers one or more drugs in the form of nanoparticles, stealth liposome, or phospholipids into the pericardium during pericardiocentesis—a procedure to drain the excess fluid, with a catheter as the non-implantable medical device. Echocardiography or ultrasound may be used to safely guide the placement of a large needle, for example, and catheter into the pericardium to deliver the one or more drugs, e.g., as nanoparticles, and to remove excess fluid. The drugs may also be delivered during a pericardial window surgical procedure.

A non-implantable medical device coated with nano-carriers may deliver drugs to many different layers in the human body, such as serous membranes (or serosae) that are smooth membranes of a thin layer of cells secreting serous fluid, and an underlying epithelial layer. For example, the non-implantable medical device coated with nano-carriers may deliver drugs to the pericardium, the serous membrane covering the heart and lining the mediastinum as described above, to the serous membrane lining the thoracic cavity and surrounding the lungs, referred to as the pleura or pleural cavity, or to the serous membrane lining the abdominopelvic cavity and viscera, referred to as the peritoneum.

In an implementation, a non-implantable medical device coated with nano-carriers delivers one or more drugs to a periosteum covering a bone in the human body. The periosteum is comprised of tough fibrous tissue which covers the external surface of bones, but can be serviced by some medical instruments.

In an implementation, a non-implantable medical device coated with nano-carriers delivers one or more drugs to a renal capsule surrounding a human kidney. The renal capsule is a tough layer of fibrous tissue, which is encased in fatty tissue. The drugs may be delivered to any of these structures, as needed for a lesion, inflammation, tumor, or disease.

In an implementation, a non-implantable medical device coated with nano-carriers delivers one or more drugs to a Glisson's capsule forming an outer covering of the human liver.

In an implementation, a non-implantable medical device coated with nano-carriers delivers one or more drugs to an articular capsule enclosing a human joint.

In an implementation, a non-implantable medical device coated with nano-carriers delivers one or more drugs to a synovial sheath enclosing a human tendon.

In an implementation, a non-implantable medical device coated with nano-carriers delivers one or more drugs to a uterine wall, such as an endometrium or inner layer of the uterine wall, the myometrium (middle layer) of the uterine wall, or to the perimetrium (outer layer) of the uterine wall.

In an implementation, a non-implantable medical device coated with nano-carriers delivers one or more drugs to other organs or encasements in the human body, such as the spleen, pancreas, intestines, and so forth.

In an implementation, a non-implantable medical device coated with nano-carriers delivers one or more drugs to a membrane or lining of the human brain or spinal cord. The nano-carriers may be delivered to one or more of the three thin layers of membrane covering the brain, called meninges. The non-implantable medical device may deliver nano-carries to the pia mater, arachnoid mater, and dura mater.

In an implementation, a non-implantable medical device coated with nano-carriers delivers one or more drugs to a myelin sheath, the fatty, white outer covering of a nerve fiber.

In an implementation, a non-implantable medical device coated with nano-carriers delivers one or more drugs to a thyroid capsule of a thyroid gland endocrine structure.

Various embodiments of the invention provide a drug-delivering instrument, such as a non-implantable balloon or catheter, that can efficiently deliver one or more drugs to a target site in the body, such as a blood vessel, organ cavity, sac, capsule, lining, layer, coating, membrane, and so forth, within a short period of time for which the drug-delivering instrument comes in contact with the target site. In addition, the invention provides drug delivery that can efficiently provide the drug to a maximum area of a lesion and provide for enhanced bioavailability with an efficient amount of the drug loaded on the drug-delivering instrument.

Those skilled in the art will realize that the above-recognized advantages and other advantages described herein are merely exemplary and are not meant to be a complete rendering of all of the advantages of the various embodiments of the invention.

In the foregoing specification, specific embodiments of the present invention have been described. However, one of ordinary skill in the art appreciates that various modifications and changes can be made without departing from the scope of the present invention as set forth in the claims below. Accordingly, the specification and figures are to be regarded in an illustrative rather than a restrictive sense, and all such modifications are intended to be included within the scope of the present invention. The benefits, advantages, solutions to problems, and any element(s) that may cause any benefit, advantage, or solution to occur or become more pronounced are not to be construed as a critical, required, or essential features or elements of any or all the claims. The present invention is defined solely by the appended claims including any amendments made during the pendency of this application and all equivalents of those claims as issued.

What is claimed is:

1. An apparatus for drug delivery, comprising:
a non-implantable medical device coated with nano-carriers for delivering one or more drugs to a body site;
a hydrophilic surface on the non-implantable medical device; and
at least one portion of the hydrophilic surface being coated with a plurality of nano-carriers.

2. The apparatus of claim 1, wherein the nano-carriers further comprise at least one drug for treating a non-significant lesion.

3. The apparatus of claim 1, wherein the nano-carriers comprise nano-carriers with different penetration capabilities for forming a reservoir of at least one drug at various different layers of a body site.

4. The apparatus of claim 3, wherein the body site comprises one of a blood vessel, an organ cavity, a sac, a capsule, a lining, a layer, a coating, a membrane, a connective tissue, or a fluid surrounding an organ.

5. The apparatus of claim 4, wherein the non-implantable medical device delivers one or more drugs into the pericardial space of a patient to prevent atherosclerosis progression, wherein the one or more drugs comprise one of nanoparticles, a stealth liposome, or phospholipids.

6. The apparatus of claim 4, wherein the body site comprises a serous membrane, a pericardium, a pleural cavity, a peritoneum, a periosteum, a renal capsule, a Glisson's capsule, an articular capsule, a synovial sheath, a meninx of a brain or spinal cord, a myelin sheath, a thyroid capsule, or a uterine wall.

7. The apparatus of claim 1, further comprising:
a first set of nano-carriers comprising a pro-healing agent;
a second set of nano-carriers comprising an anti-proliferative agent; and
a third set of nano-carriers comprising one or both of an anti-inflammatory agent and an anti-thrombogenic agent.

8. The apparatus of claim 7, wherein the first set of nano-carriers have a first average diameter ranging from 800 nm to 1500 nm;
the second set of nano-carriers have a second average diameter ranging from 300 nm to 800 nm; and
the third set of nano-carriers have a third diameter ranging from 10 nm to 300 nm.

9. The apparatus of claim 8, wherein a first time required for the third set of nano-carriers to release from the hydrophilic surface upon coming in proximity of the body site is less than a second time required for the second set of nano-carriers and the first set of nano-carriers to release from the hydrophilic surface;
wherein the third set of nano-carriers exhibit a rapid rate of release from the hydrophilic surface and wherein the second set of nano-carriers and the first set of nano-carriers exhibit slower rates of release from the hydrophilic surface as compared with the rate of release of the third set of nano-carriers.

10. The apparatus of claim 9, wherein at least one adjacent portion of the hydrophilic surface is uncoated and exposable to a blood or a tissue environment for rapid dissolution from the non-implantable medical device and for rapid dissolution from underneath the nano-carriers of an adjacent coated portion of the hydrophilic surface, the rapid dissolution from underneath providing an increase in the rapidity of release of the nano-carriers of the adjacent coated portion;
each nano-carrier of the plurality of nano-carriers comprising a respective agent surrounded by an encapsulating medium; and
wherein a surface of each nano-carrier is devoid of the respective agent, and 30% to 80% of the plurality of the nano-carriers are released from the hydrophilic surface within 15-90 seconds when the non-implantable medical device comes in proximity to the body site.

11. The apparatus of claim 1, wherein the encapsulating medium comprises at least one of a biological agent, a phospholipid and a blood excipient.

12. The apparatus of claim 11, wherein the at least one biological agent is selected from a group comprising drug carriers, excipients, blood components, excipients derived from blood, phospholipids, solid lipid nano-particles, lipoids, vitamins and sugar molecules.

13. The apparatus of claim 11, wherein the biological agent is selected from a group comprising steroids, vitamins, estradiol, esterified fatty acids, non-estrified fatty acids, glucose, inositol, L-lactate, lipoproteins, carbohydrates, tricalcium phosphate, precipitated calcium phosphate, calcium phoshate tribasic, substances derived from at least one of human, egg and soybean, phospholipon 80H, phospholipon 90H, Lipoids S75, Lipoids E80, Intralipid 20, Lipoid EPC, Lipoids E75, lipids obtained from egg, lipids obtained from soya, phosphatidylcholine, phosphatidylglycerol, phosphatidylinositol, phosphatidylserine, phosphatidic acid, cardiolipin, and phosphatidylethanolamine.

14. The apparatus of claim 1, wherein one of the drugs is selected from a group consisting of anti-proliferative agents, anti-inflammatory agents, anti-neoplastic agents, anti-coagulant agents, anti-fibrin agents, antithrombotic agents, anti-mitotic agents, antibiotic agents, anti-allergic agents and antioxidants, at least one flavonoid, estrogen, protease inhibitors, antibodies, immunosuppressive agents, cytostatic agents, cytotoxic agents, calcium channel blockers, phosphodiesterase inhibitors, prostaglandin inhibitors, dietary supplements, vitamins, anti-platelet aggregating agents and genetically engineered epithelial cells, wherein the at least one flavonoid is selected from a group comprising at least one of narigenin, naringin, eriodictyol, hesperetin, hesperidin (esperidine), kampferol, quercetin, rutin, cyanidol, meciadonol, catechin, epi-gallocatechin-gallate, taxifolin (dihydroquercetin), genistein, genistin, daidzein, biochanin, glycitein, chrysin, diosmin, luetolin, apigenin, tangeritin and nobiletin.

15. The apparatus of claim 1, wherein one of the drugs is selected from a group consisting of sirolimus, paclitaxel, tacrolimus, clobetasol, dexamethasone, genistein, heparin, beta-estadiol, rapamycin, everolimus, ethylrapamycin, zotarolimus, ABT-578, Biolimus A9, docetaxel, methotrexate, azathioprine, vincristine, vinblastine, fluorouracil, doxorubicin hydrochloride, mitomycin, sodium heparin, low molecular weight heparin, heparinoid, hirudin, argatroban, forskolin, vapiprost, prostacyclin, prostacyclin analogues, dextran, D-phe-pro-arg-chloromethylketone, dipyridamole, glycoprotein IIb/IIIa, recombinant hirudin, bivalirudin, nifedipine, colchicines, lovastatin, nitroprusside, suramin, serotonin blockers, a steroid, thioprotease inhibitors, triazolopyrimidine, nitric oxide, nitric oxide donors, super oxide dismutase, super oxide dismutase mimetics, estradiol, aspirin, angiopeptin, captopril, cilazapril, lisinopril, permirolast potassium, alpha-interferon, and bioactive RGD.

16. The apparatus of claim 1, wherein one of the drugs is selected from a group consisting of sirolimus and paclitaxel.
